(19) United States
(12) Patent Application Publication
(10) Pub. No.: US 2023/0331771 A1
(43) Pub. Date: Oct. 19, 2023

(54) METHOD FOR PREPARING ATELOCOLLAGEN PREPARED IN HIGH PURITY AND HIGH YIELD AND USE THEREOF

(71) Applicant: UNION KOREA LIFE SCIENCES INC., Jiwang-si, Gyeonggi-do (KR)

(72) Inventors: Jung Seok KIM, Uijeongbu-si Gyeonggi-do (KR); Bying Ha BACK, Seongnam-si Gyeonggi-do (KR)

(21) Appl. No.: 18/025,583

(22) PCT Filed: Sep. 9, 2021
(86) PCT No.: PCT/KR2021/012266

§ 371 Date: Mar. 9, 2023

(30) Foreign Application Priority Data
Sep. 10, 2020 (KR) 10-2020-0115837
Sep. 6, 2021 (KR) 10-2021-0118419

(51) Int. Cl. C07K 1/12; C07K 14/78; C07K 1/36; C09D 11/04; A61L 27/24

(52) U.S. Cl. CPC C07K 1/12; C07K 14/78; C07K 1/36; C09D 11/04; A61L 27/24

(57) ABSTRACT

Provided are a method of preparing atelocollagen and use thereof, more specifically, a method of removing immunogens from pig skin-derived collagen and preparing high-purity atelocollagen with high yield, and use thereof. Atelocollagen prepared with high yield, according to a preparation method of the present disclosure, has high purity, a low immune response, no cytotoxicity, excellent in vivo biodegradability, and high safety, and therefore, may be used in a form of a bio-ink capable of forming high-resolution structures with excellent morphological characteristics and division ability, and therefore, is expected to be used for the development of a product for tissue and regeneration engineering, including a cell scaffold capable of easily forming blood vessels and controlling drug delivery.

### TECHNICAL FIELD

The present disclosure relates to a method of preparing atelocollagen and use thereof, more specifically, to a method of removing immunogens from pig skin-derived collagen and preparing high-purity atelocollagen with high yield, and use thereof.

### BACKGROUND ART

Collagen is a main protein component of the extracellular matrix, and is very abundant in soft tissues including connective tissues such as skin, tendon, and blood vessels, as well as hard tissues such as bones and teeth, and collagen accounts for about ⅓ of the total protein in mammals and plays a role in forming basic structures of tissues or organs by assembling cells in a certain order. Without collagen, multicellular animals cannot exist. Therefore, when trying to regenerate a damaged part of a living body into its original tissue, the tissue cells will have regenerative power when the extracellular matrix of the tissue is supplied to the damaged part, and therefore, providing collagen as a basic matrix for artificial tissue substitutes may be very useful.

On the other hand, although there are many tissues containing collagen in the living body, such as skin, ligaments, bones, blood vessels, amnion, pericardium, heart valves, placenta, and cornea, the type of collagen is different in each tissue. In particular, type 1 collagen is most widely used in tissue engineering because it is contained in large amounts in almost all tissues such as skin, ligaments, and bones. However, at both ends of a collagen molecule, there is a portion called a telopeptide that does not form a helix, which is a main cause of an immune response, and thus, it is recommended that atelocollagen from which this part has been removed is used when using collagen as a raw material for medicines or cosmetics.

Atelocollagen refers to collagen in which telopeptides at the ends of the collagen molecule that cause immune responses are removed from normal collagen. While normal collagen induces an immune response of about 20%, atelocollagen induces an immune response of less than 0.3% and can be used as a biocompatible material, and therefore, it is used as a variety of biomaterials.

Accordingly, various methods of producing atelocollagen with immunogenicity removed have been studied (Korean Patent Publication No. 10-2017-0055645), but the yield is low, so it is urgent to develop a method that increases the production yield and purity of atelocollagen.

### DISCLOSURE

### Technical Problem

As a result of endeavoring to produce atelocollagen with an improved yield, the present inventors completed the present disclosure by confirming that high-purity atelocollagen may be produced with high yield through a change in a pig skin pre-treatment process.

Accordingly, an object of the present disclosure is to provide a method of preparing high-purity atelocollagen with high yield, including: (a) pre-treating pig skin by washing it, treating it with acetic acid to remove fat, and stirring it with ethanol;
(b) treating the pre-treated pig skin with pepsin to remove telopeptides of the pig skin to obtain collagen with immunogenicity removed;
(c) washing the collagen with immunogenicity removed with ethanol, and then separating it by centrifugation to obtain intermediate atelocollagen;
(d) treating the intermediate atelocollagen with urea;
(e) purifying and concentrating the intermediate atelocollagen treated with urea; and
(f) removing moisture from the purified and concentrated atelocollagen by lyophilization.

In addition, an additional object of the present disclosure is to provide atelocollagen prepared by the above preparation method, and a bio-ink composition including the atelocollagen.

In addition, still another object of the present disclosure is to provide a cell scaffold including the bio-ink composition.

However, technical problems to be achieved by the present disclosure is not limited to the above-mentioned problems, and other problems not mentioned will be clearly understood by those skilled in the art from the following description.

### Solution to Problem

In order to achieve the above-described objects, the present disclosure provides a method of preparing high-purity atelocollagen with high yield, including: (a) pre-treating pig skin by washing it, treating it with acetic acid to remove fat, and stirring it with ethanol;
(b) treating the pre-treated pig skin with pepsin to remove telopeptides of the pig skin to obtain collagen with immunogenicity removed;
(c) washing the collagen with immunogenicity removed with ethanol, and then separating it by centrifugation to obtain intermediate atelocollagen
(d) treating the intermediate atelocollagen with urea;
(e) purifying and concentrating the intermediate atelocollagen treated with urea; and
(f) removing moisture from the purified and concentrated atelocollagen by lyophilization.

In an embodiment of the present disclosure, the present disclosure may further include a storage process of storing the pre-treated pig skin frozen at a low temperature of −30° C. or less after the process (a).

In an embodiment of the present disclosure, the present disclosure may further include a process of homogenizing after blending the pre-treated pig skin after the storage process.

In another embodiment of the present disclosure, the present disclosure may repeatedly perform process (b) 2 times to 5 times.

In another embodiment of the present disclosure, the present disclosure may further include performing salting-out filtration before proceeding with ethanol washing in the process (c).

In another embodiment of the present disclosure, the process of purifying and concentrating in the process (e) may use tangential flow filtration.

In addition, the present disclosure provides atelocollagen prepared by the preparation method.

In addition, the present disclosure provides a bio-ink composition including the atelocollagen or a cell scaffold including the bio-ink composition.

### Advantageous Effects

Atelocollagen, prepared with high yield according to a preparation method of the present disclosure, has high purity, a low immune response, no cytotoxicity, excellent in vivo biodegradability, and high safety, and therefore, may be used in a form of a bio-ink capable of forming high-resolution structures with excellent morphological characteristics and division ability, and therefore, is expected to be used for development of a product for a tissue and regeneration engineering, including a cell scaffold capable of easily forming blood vessels and controlling drug delivery.

### DESCRIPTION OF DRAWINGS

FIG. 1 relates to a method of producing atelocollagen according to the present disclosure, FIG. 1A shows a schematic diagram of atelocollagen preparation processes, and FIG. 1B shows photographs of atelocollagen extraction and purification processes.

FIG. 2 shows a schematic diagram of atelocollagen prepared by the present disclosure.

FIG. 3 is results of an analysis of quality of atelocollagen prepared by the present disclosure, in which FIG. 3A shows Sircol kit results, FIG. 3B shows results of bicinchoninic acid assay (BCA) protein assay, and FIG. 3C shows results of sodium dodecyl sulfate-polyacrylamide gel electrophoresis (SDS-PAGE) molecular weight analysis.

FIG. 4 shows results of production yield (%) of atelocollagen prepared by the present disclosure.

FIG. 5 shows purity (%) of atelocollagen prepared by the present disclosure.

FIG. 6 is a diagram showing cell viability according to treatment with atelocollagen prepared according to the present disclosure and a commercially available product (Comparative Example F).

FIG. 7 is a diagram showing results confirmed by staining cells treated with atelocollagen prepared according to the present disclosure and a commercially available product (Comparative Example F).

FIGS. 8A to 8G are results of confirming in vivo biodegradability by transplanting atelocollagen prepared by the present disclosure into the body.

FIG. 9 is results of confirming changes in body weight of mice when atelocollagen prepared according to the present disclosure is transplanted into the body.

FIG. 10 shows results of scanning electron microscope (SEM) imaging of a structure prepared by using an atelocollagen-based bio-ink prepared according to the present disclosure.

FIG. 11 shows results of confirming cell viability in an atelocollagen-based bio-ink prepared according to the present disclosure.

FIG. 12 shows results of analyzing proliferation rates of cells after coating with atelocollagen, in order to confirm biological properties of an atelocollagen-based bio-ink prepared according to the present disclosure.

### MODE FOR INVENTION

Hereinafter, the present disclosure will be described in more detail.

As a result of endeavoring to produce atelocollagen with an improved yield, the present inventors completed the present disclosure by confirming that high-purity atelocollagen may be produced with high yield through a change in a process such as pig skin pre-treatment.

Accordingly, the present disclosure provides a method of preparing high-purity atelocollagen with high yield, including: (a) pre-treating the pig skin by washing it, treating it with acetic acid to remove fat, and stirring it with ethanol;
(b) treating the pre-treated pig skin with pepsin to remove telopeptides of the pig skin to obtain collagen with immunogenicity removed;
(c) washing the collagen with immunogenicity removed with ethanol, and then separating it by centrifugation to obtain intermediate atelocollagen
(d) treating the intermediate atelocollagen with urea;
(e) purifying and concentrating the intermediate atelocollagen treated with urea; and
(f) removing moisture from the purified and concentrated atelocollagen by lyophilization.

The present disclosure may further include a storage process of storing the pre-treated pig skin frozen at a low temperature of −30° C. or less after the process (a).

In the present disclosure, in process (b), pepsin may be treated once or more times at a concentration of 0.5×10⁸ units to 2.0×10⁸ units per 1 kg of pig skin. It is preferable to additionally perform a homogenization process after the pepsin treatment and then to filter the pepsin.

In the present disclosure, washing with ethanol in the process (c) may be performed once or more times by using 1 L to 20 L of ethanol per 1 kg of pig skin, and may further include performing salting-out filtration before proceeding with the washing with ethanol.

In the present disclosure, in process (d), urea may be treated at 0.01 M to 0.03 M.

In the present disclosure, the process of purifying and concentrating in process (e) may use tangential flow filtration.

According to a preparation method of the present disclosure high-purity atelocollagen may be prepared with high yield, and atelocollagen exhibiting a uniform molecular weight of 120 M.W to 380 M.W may be prepared.

Hereinafter, the atelocollagen preparation method of the present disclosure will be described in detail subdivided into each process.

[Process (a): Pre-Treatment of Pig Skin]

As a pre-treatment process for preparing the dermis of pig skin, HACCP-compatible pig skin is swollen, washed, and then treated with acetic acid to remove physical fat, and is sterilized. The sterilized pig skin is stirred in ethanol and blended and then a homogenization process is applied.

[Process (b): Removal of Immunogenicity]

In this process, in order to remove telopeptides at the ends of collagen molecules that cause an immune responses in the pre-treated pig skin, a specific concentration of pepsin is treated and inactivated to produce collagen with immunogenicity removed. The pepsin may be treated repeatedly, and a homogenization process is additionally performed after each pepsin treatment, and a pepsin filtration process is performed after each treatment.

[Process (c): Obtaining Atelocollagen]

The collagen with immunogenicity removed is washed several times with ethanol, and is subjected to step-by-step filtration, and then centrifuged, and after removing fat and impurities from the upper and lower layers, a middle layer is obtained. From the obtained middle layer, atelocollagen is extracted by salting-out reactions.

[Process (d): Urea Treatment and Separation and Purification]

In this process, the atelocollagen obtained in Process (c) is treated with urea, then sterilized, and separated and purified by using tangential flow filtration.

[Process (e): Removal of Moisture]

In this process, the atelocollagen separated and purified in Process (d) is lyophilized to obtain atelocollagen from which moisture is removed.

The present inventors have identified efficacy of high-purity atelocollagen prepared with high yield by the preparation method of the present disclosure through specific examples.

In an example of the present disclosure, the preparation method of atelocollagen according to the present disclosure (refer to Example 1-1) was investigated, and as a result of analyzing a production yield, molecular weight, and purity of atelocollagen produced by the preparation method of the present disclosure, productivity increased by twofold or more compared to the existing process, confirming an efficacy improved by 400% compared to the current technology, and in terms of purity, it was confirmed that purity (98.4%) was significantly improved compared to the control atelocollagen (86.7%) prepared by the existing process (see Example 1-3).

In addition, as a result of evaluating cytotoxicity of atelocollagen prepared by the preparation method of the present disclosure, it was confirmed that the cytotoxicity was lower than that of commercially available products (see Example 1-4), and as a result of evaluating in vivo biodegradability and safety, it was confirmed that the in vivo biodegradability and safety were also excellent (see Example 1-5).

From the above results, it was confirmed that the atelocollagen produced by the preparation method of the present disclosure has high purity and exhibit a high production yield, and may be used for development of a product for a tissue and regeneration engineering, including a cell scaffold.

Accordingly, as another aspect of the present disclosure, the present disclosure provides atelocollagen prepared by the above preparation method and a bio-ink composition including the same.

The term “bio-ink”, used herein, refers to all materials including living cells or biomolecules, that may be applied to bioprinting technology to produce required structures. The term “bio-ink” refers to a material that provides physical properties for 3D processing and a biological environment for cells to perform a desired function, and has excellent cell affinity.

The bio-ink of the present disclosure may be applied to various types of 3D printers that can print bio-structures that may be used in the medical- and bio-fields, such as scaffolds for tissue engineering, surgical implants, personalized implants, artificial blood vessels, and artificial organs, through 3D printers, but is not limited thereto.

In addition, the present disclosure provides a cell scaffold including the bio-ink composition.

The cell scaffold according to the present disclosure may be a single tissue structure capable of being implanted in the body, or a tissue structure with improved functionality capable of reproducing tissue-to-tissue association by simultaneously printing several cells.

Hereinafter, preferred examples are presented to aid understanding of the present disclosure. However, the following examples are provided for an easier understanding the present disclosure, and the content of the present disclosure is not limited by the following examples.

### EXAMPLE

### Example 1

### Extraction of Atelocollagen and Confirmation of its Characteristics

1-1. Atelocollagen Preparation Process

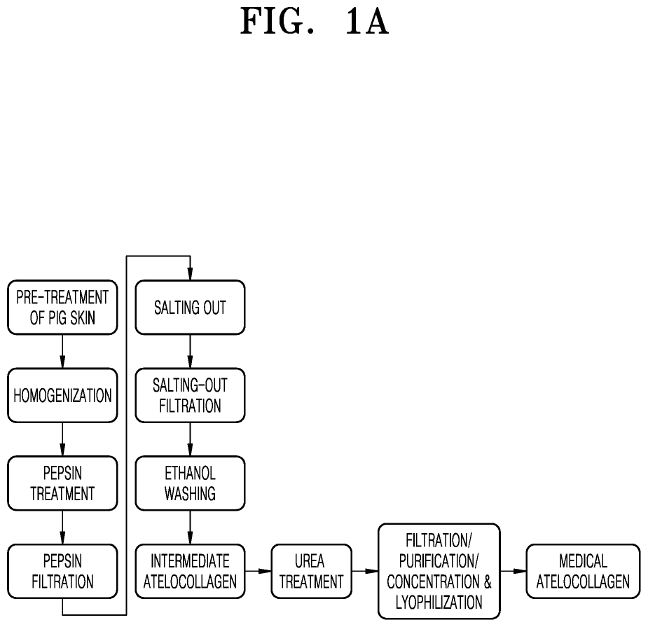

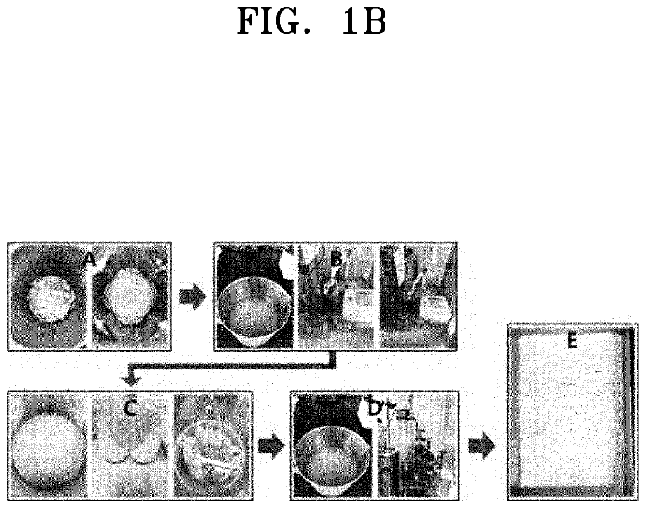

In order to extract atelocollagen from pig skin, atelocollagen was extracted by preparation processes shown in FIGS. 1A and 1B. Specifically, the following processes were performed.

First, pig skin was pretreated as follows: after swelling and washing HACCP-certified pig skin, 0.1 M to 1.0 M acetic acid was treated overnight to remove physical fat, and the sterilized pig skin was stirred in ethanol overnight. Thereafter, it was stored frozen at a cryogenic temperature of −30° C. or less.

Thereafter, the frozen pig skin stored at a cryogenic temperature was re-swollen and subjected to an acetic acid removal processes, blended, and then processed through a homogenizing process.

Next, 0.5×10⁸ units to 2.0×10⁸ units of pepsin per 1 kg of pretreated frozen pig skin was treated at a temperature of 1° C. to 10° C. for 24 hours to remove telopeptides which are at the ends of collagen molecules and cause immune responses, followed by pepsin filtration to inactivate pepsins, and the above process was repeated several times to remove the telopeptides as much as possible.

Next, the pepsin-inactivated collagen was salted out overnight at a temperature of 1° C. to 10° C. by using 2.5 M to 5.0 M of NaCl per 1 kg of pig skin, and salting-out filtration was performed through centrifugation.

Thereafter, the mixture was washed several times by using 1 L to 20 L of ethanol, subjected to step-by-step filtration treatment overnight at a temperature of 1° C. to 10° C., and then centrifuged. Atelocollagen was extracted from the middle layer obtained by removing fat and impurities from the upper and lower layers resulting from the centrifugation.

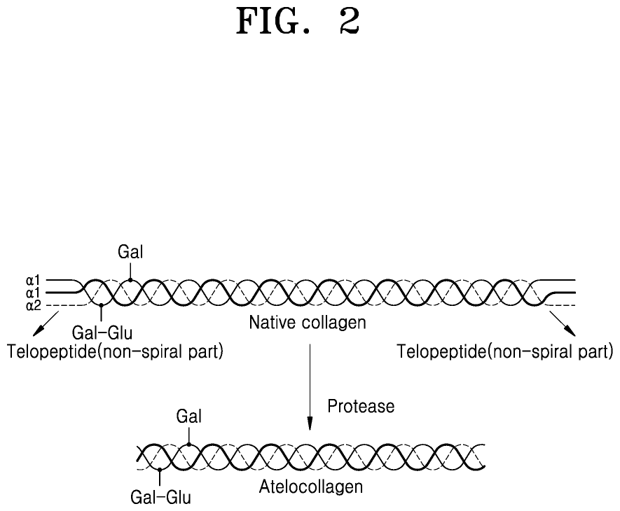

Next, the obtained atelocollagen was treated with 0.01 M to 0.03 M of urea, sterilized overnight at a temperature of 1° C. to 10° C., and then separated and purified by using tangential flow filtration. The separated and purified atelocollagen was lysophilized to remove moisture, and atelocollagen of a uniform molecular weight of 120 M.W to 380 M.W was prepared. A schematic diagram of the atelocollagen prepared in the present disclosure is shown in FIG. 2.

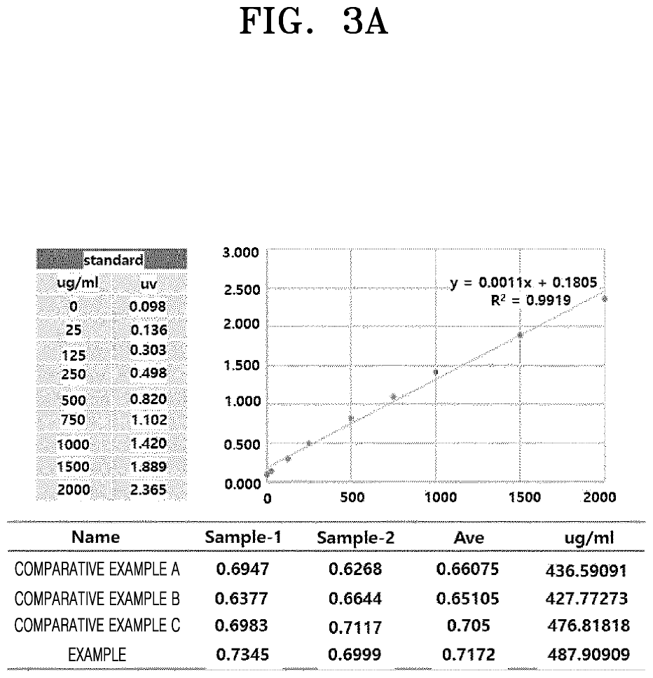

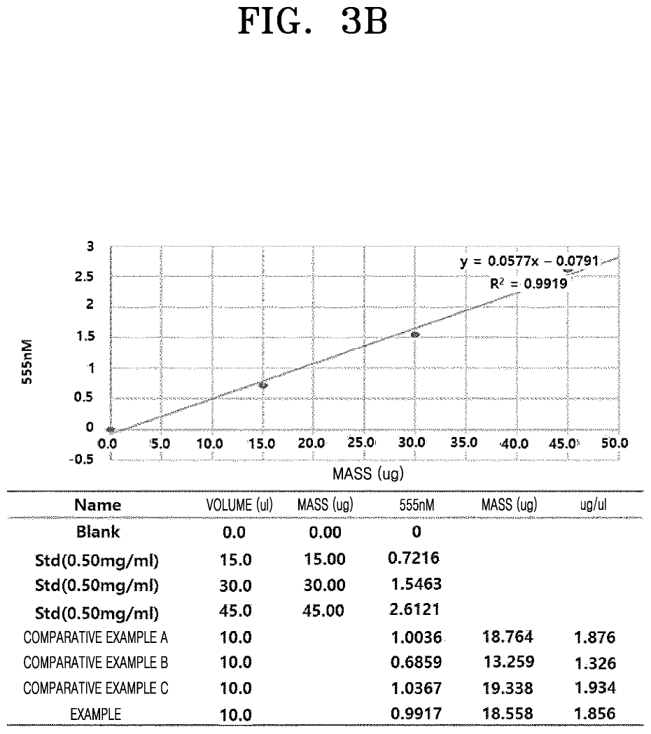

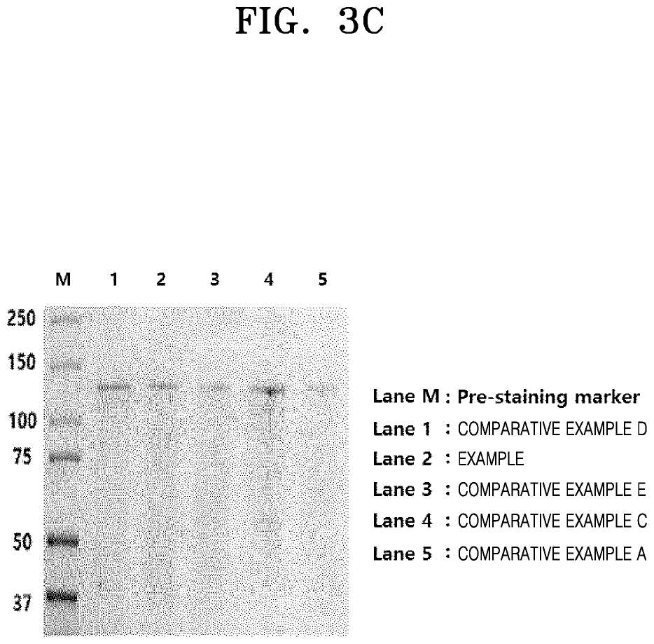

Sircol kit analyses (FIG. 3A), bicinchoninic acid assay (BCA) protein analyses (FIG. 3B), and sodium dodecyl sulfate-polyacrylamide gel electrophoresis (SDS-PAGE) molecular weight analyses (FIG. 3C) were performed for a product prepared by the preparation method (Example), and commercially available products (Comparative Examples A, B, C, D and E), in order to confirm quality of the prepared atelocollagen. As a result, as shown in FIG. 3A, when production yields were compared by using the Sircol Kit, it was confirmed that an amount of newly synthesized collagen was greater in the example compared to the comparative examples, and as shown in FIGS. 3B and 3C, the collagen of the example showed quality equal to that of currently commercially available high-purity standard collagen for medical use, regenerative medicine, and research, or products of other companies.

In addition, as a result of identifying the quality through an internal quality evaluation and an institution for certification and evaluation, it was confirmed that the quality of the atelocollagen prepared according to the example was excellent.

1-2. Preparation Process of Comparative Examples

First, pig skin was pretreated by the following process: after swelling and washing pig skin, the physical fat was removed, and the sterilized pig skin was stirred in ethanol, blended, and then homogenized. Afterwards, it was stored in a general freezing condition of a temperature of −20° C.

Next, 1×10⁸ units to 2×10⁸ units of pepsin per 1 kg of the frozen pig skin was treated once or twice for 6 hours or overnight to remove the telopeptides at the ends of collagen molecules that cause immune responses.

Next, the pepsin-inactivated collagen was washed with ethanol in an amount sufficient to submerge the pig skin, and filtration was performed overnight at 4° C., followed by centrifugation, and from the middle layer obtained by removing fat and impurities from the upper and lower layers, atelocollagen was extracted.

Next, the obtained atelocollagen was treated with 0.01 M to 0.05 M urea and sterilized, and then separated and purified to prepare atelocollagen showing various M.V.

1-3. Confirmation of Yield and Purity of Atelocollagen

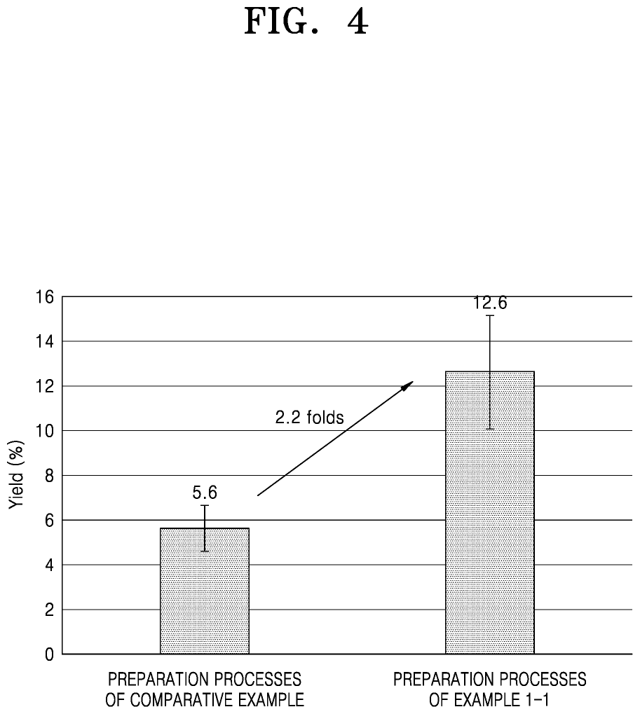

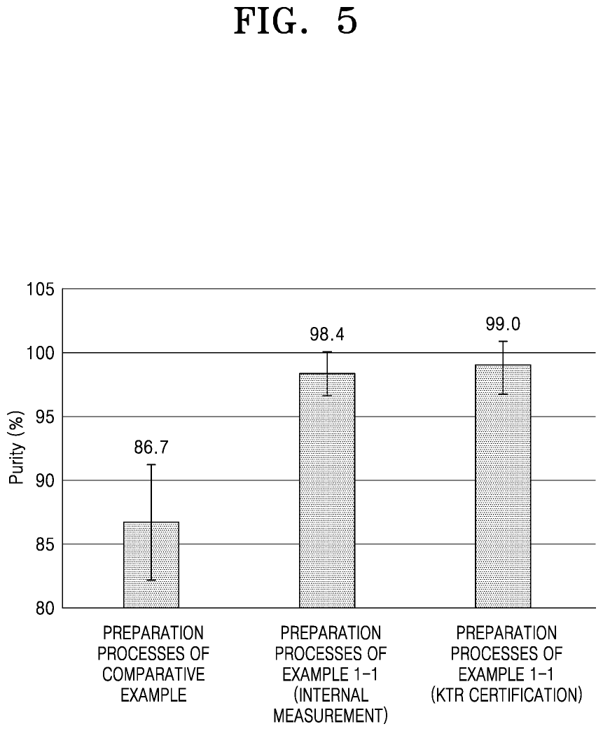

A production yield, molecular weight and purity of atelocollagen prepared by the method of Example 1-1 were analyzed by using a Sircol kit and SDS-PAGE, and the results are shown in Table 1, FIGS. 4 and 5.

TABLE 1

| Key | | Atelocollagen | Level of the | |
|---|---|---|---|---|
| performance | | of the present | existing | Measurement |
| indicators | Unit | disclosure | technology | method |
| Production | % | 12.6% ± 2.5 | 4% | Sircol kit |
| yield | | | | |
| Molecular | kDa | 240 kDa or | 240 kDa or | SDS-PAGE |
| weight | | more | more | |
| Purity | % | 99% ± 2.1 | 99% | Sircol kit |

From the results of Table 1, it was confirmed that atelocollagen prepared in the present disclosure showed a significantly excellent yield (12.6%) compared to the previous highest production yield of 4%, and it was confirmed that the molecular weight and purity were similar to the previous highest level.

In addition, as shown in FIG. 4, it was confirmed that the production yield and productivity of atelocollagen prepared according to the preparation process of the present disclosure are increased by more than two times compared to atelocollagen of a comparative example prepared by a process in the art.

In addition, as shown in FIG. 5, it was confirmed that purity (98.4%) of atelocollagen prepared according to the preparation process of the present disclosure was significantly improved compared to purity (86.7%) of atelocollagen of a comparative example prepared by an existing process. In particular, as a result of confirming purity (99%) through a certification evaluation institution, the test results were shown to be similar.

1-4. Atelocollagen Cytotoxicity Assessment

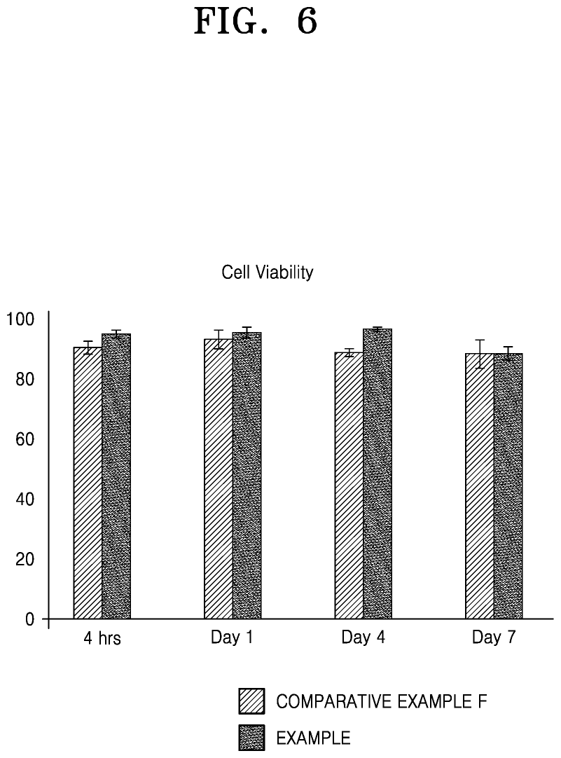

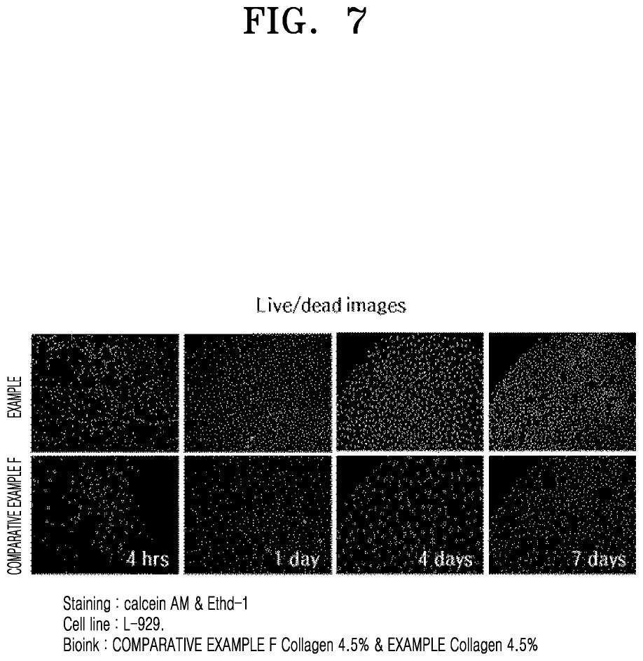

Cytotoxicity of atelocollagen prepared by the method of Example 1-1 was compared with that of a commercially available product (Comparative Example F), and the results are shown in FIGS. 6 and 7.

Specifically, after preparing an aqueous solution of pH 7.0 to pH 7.5 of 4.5% atelocollagen of a commercially available product (Comparative Example F) and of an Example, L-929 cells were mixed in the atelocollagen aqueous solution to a concentration of 1×10⁶ cells/ml, and cell viability was evaluated for 4 hours, 1 day, 4 days, and 7 days, and after staining the cells with a CCK-8 solution, optical density (OD) values at 450 nm were measured and analyzed by using a microplate reader (GloMax; Promaga, USA), and are shown in FIG. 6.

In addition, FIG. 7 is results of conducting LIVE/DEAD assays for cell viability analysis, and cells cultured by the same test method as in FIG. 6 are stained by using a LIVE/DEAD staining solution including calcein AM and EthD-1. Fluorescence-stained cell samples were imaged and observed by using a confocal microscope (FV1200; Olympus, Japan).

Specifically, L-929 cell lines were treated with atelocollagen (Example and Comparative Example F) at a concentration of 4.5%, and cytotoxicity was confirmed after 4 hours, on day 1, day 4, and day 7. As a result, as shown in FIG. 6, the commercial product (Comparative Example F) showed cytotoxicity of an average of 10% for each time, whereas the atelocollagen of the Example showed cytotoxicity of an average of 5%, and it was confirmed that the atelocollagen prepared according to the example showed lower cytotoxicity compared to the commercial product (Comparative Example F).

In addition, the L-929 cell lines were treated with atelocollagen at a concentration of 4.5% (Example and Comparative Example F), and calcein AM and Ethd-1 staining were performed to confirm cytotoxicity after 4 hours, on day 1, day 4, and day 7, and the results are shown in FIG. 7. As shown in FIG. 7, it was confirmed that atelocollagen of the Example had a large number of live cells compared to the commercially available product (Comparative Example F).

1-5. Evaluation of In Vivo Biodegradability and Stability of Atelocollagen

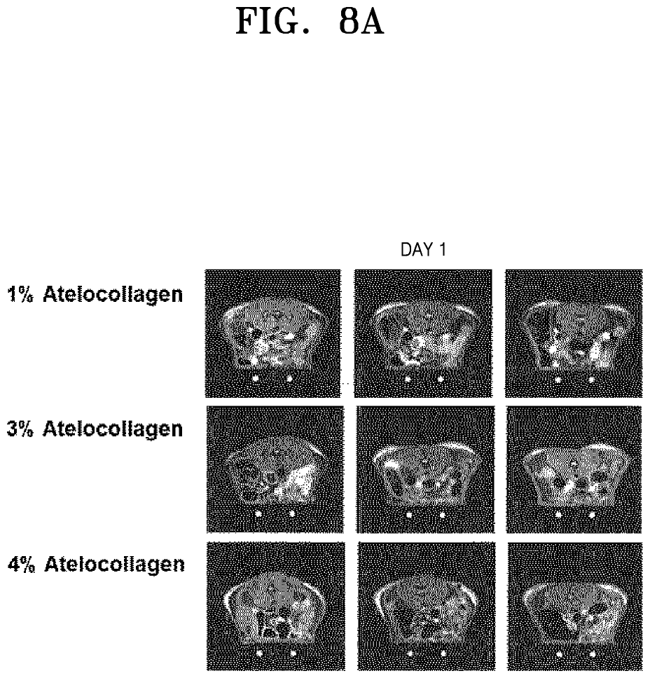

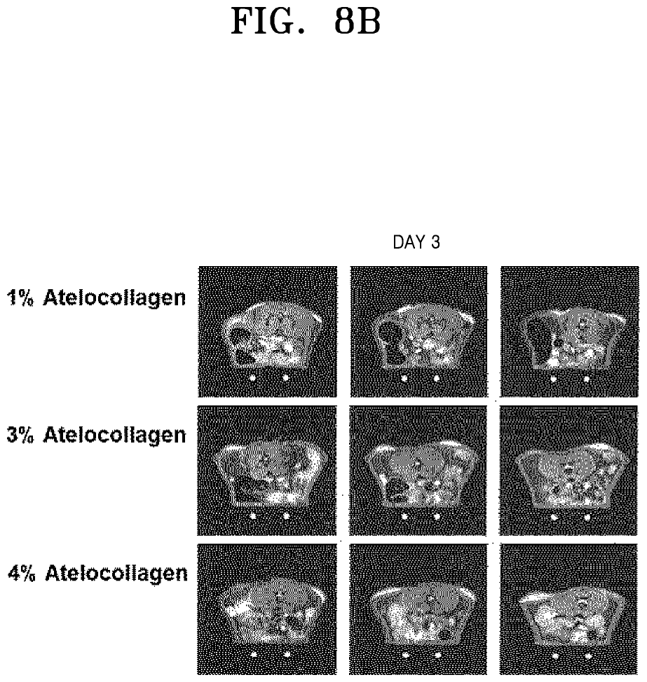

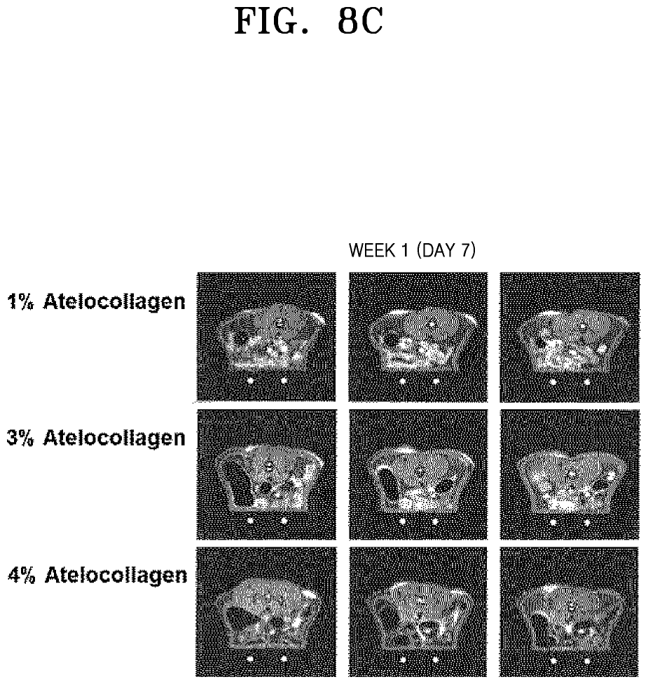

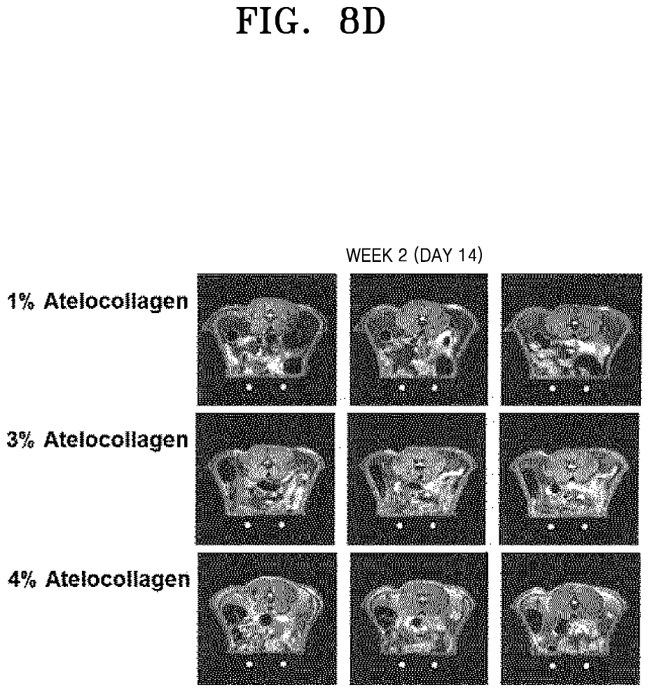

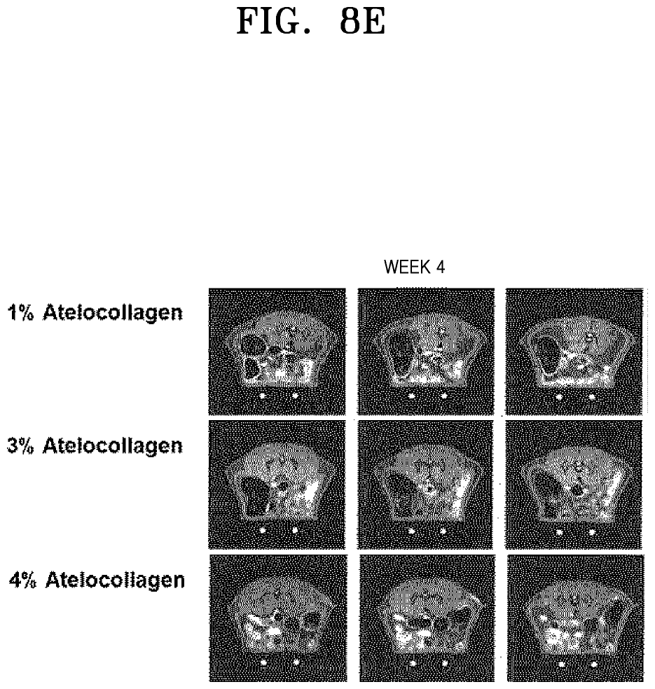

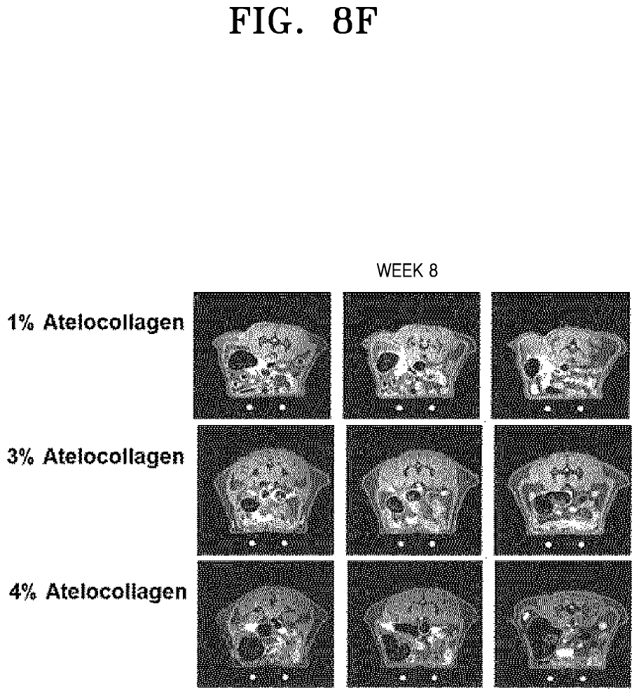

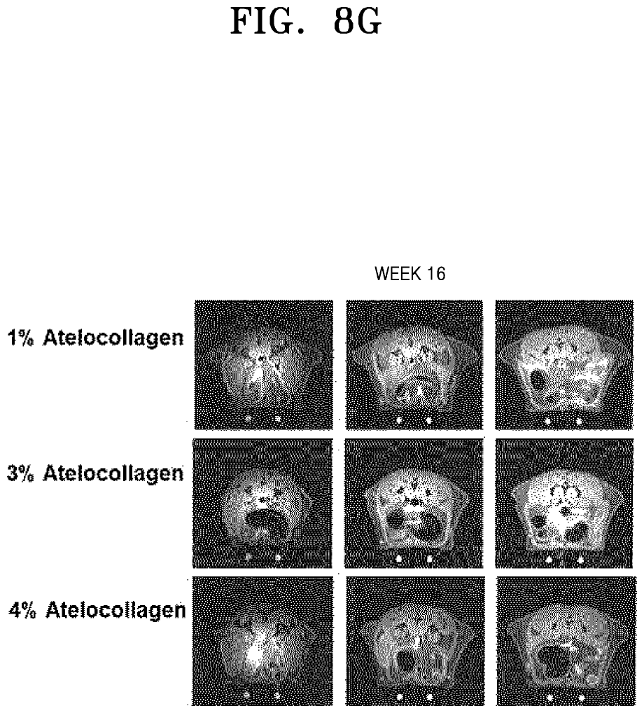

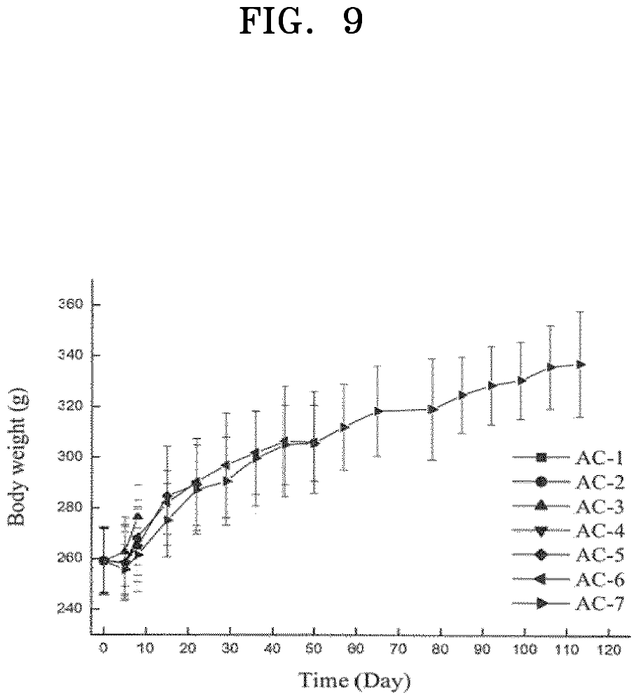

In vivo biodegradability and safety of atelocollagen prepared by a method of Example 1-1 were evaluated, and the results are shown in FIGS. 8A to 8G, and 9.

Specifically, atelocollagen of each concentration (1%, 3%, and 4%) was prepared by the method of Example 1-1, trasplanted into mice, and liver biodegradability was confirmed for 16 weeks, through a public certification evaluation institution.

Specifically, an evaluation of biodegradability and stability of atelocollagen prepared by the method of Example 1-1 was requested to a public certification evaluation institution. For a test evaluation of FIGS. 8A to 8G and 9, rats (SD Rat, Orient Bio, Korea), which are rodents, were used as experimental animals, and 0.25 g of atelocollagen aqueous solutions prepared at each concentration (1%, 3%, and 4%) were injected subcutaneously on both sides of the back based on the spinal line. Body weight was measured before administration, on the day of administration, and once a week as an item for confirming toxicity after transplantation. For MRI imaging, MR imaging equipment (BioSpec; Bruker, Germany) was used to take images of each subject from day 1 to week 16 according to the experimental schedule, and the images were analyzed.

As a result of confirming through MR imaging, it was confirmed that the decomposition was concentration-dependent (FIGS. 8A to 8G). It was confirmed that biodegradation was concentration-dependent, and in particular, when 1% and 3% atelocollagen was transplanted, the atelocollagen was confirmed to be degraded within 1 week to 2 weeks, and when 4% atelocollagen was transplanted, the atelocollagen was confirmed to be degraded between 3 weeks and 4 weeks.

In addition, it was confirmed that there was no weight loss in the animals transplanted with atelocollagen, and therefore, it was confirmed that there was no toxicity (FIG. 9).

### Example 2

### Preparation of Bio-Ink Using High-Purity Atelocollagen and Confirmation of its Characteristics

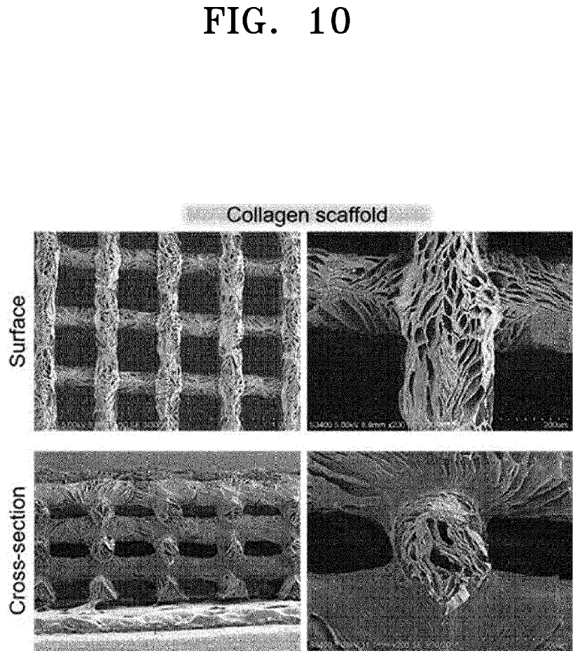

A bio-ink was prepared from pig skin-derived collagen from which immunogens have been removed/atelocollagen and extracellular matrix by using a cross-linking method using a double hydrogen bonding reaction. Scanning electron microscope (SEM) images were taken as shown in FIG. 10, to examine feasibility of implementing a target resolution and characteristics of a structure prepared by using a 3D bioprinter and an atelocollagen-based bioink.

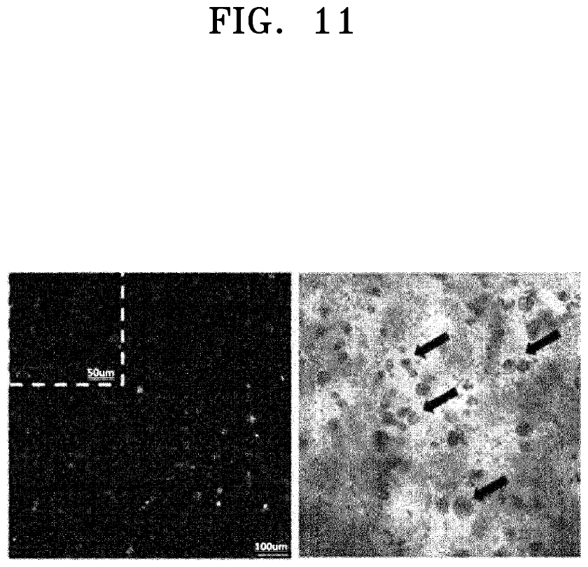

In addition, as a result of confirming cell viability to evaluate the feasibility of 3D bio-ink for medical and regenerative use, as shown in FIG. 11, cell attachment and viability were confirmed in the atelocollagen-based structure.

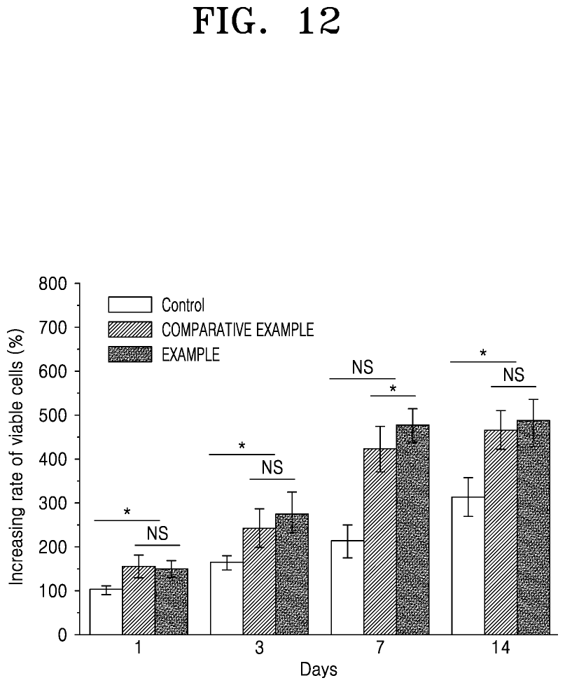

In addition, as a result of analyzing increasing rates of viable cells (%) for 1 day to 14 days after coating with a control, commercially available atelocollagen (Comparative Example) and atelocollagen (Example) of the present disclosure, in order to confirm cell morphology and division after coating with atelocollagen, it was confirmed that a proliferation rate of cells coated with atelocollagen of the present disclosure was higher than that of atelocollagen of other companies after day 3, as shown in FIG. 12.

The above description of the present disclosure is for illustrative purposes, and those skilled in the art to which the present disclosure belongs will be able to understand that the examples and embodiments can be easily modified without changing the technical idea or essential features of the disclosure. Therefore, it should be understood that the above examples are not limitative, but illustrative in all aspects.

1. A method of preparing high-purity atelocollagen with high yield, comprising:
(a) pre-treating pig skin by washing it, treating it with acetic acid to remove fat, and stirring it with ethanol;
(b) treating the pre-treated pig skin with pepsin to remove telopeptides of the pig skin to obtain collagen with immunogenicity removed;
(c) washing the collagen with immunogenicity removed with ethanol, and then separating it by centrifugation to obtain intermediate atelocollagen;
(d) treating the intermediate atelocollagen with urea;
(e) purifying and concentrating the intermediate atelocollagen treated with urea; and
(f) removing moisture from the purified and concentrated atelocollagen by lyophilization.

2. The method of claim 1, further comprising a storage process of storing the pre-treated pig skin frozen at a low temperature of −30° C. or less after the process (a).

3. The method of claim 2, further comprising a process of homogenizing after blending the pre-treated pig skin after the storage process.

4. The method of claim 1, wherein the process (b) is repeated 2 times to 5 times.

5. The method of claim 1, further comprising performing salting-out filtration before proceeding with ethanol washing in process (c).

6. The method of claim 1, wherein the process of purifying and concentrating in the process (e) uses tangential flow filtration.

7. Atelocollagen prepared by the method of claim 1.

8. A bio-ink composition, comprising the atelocollagen of claim 7.

9. A cell scaffold comprising the bio-ink composition of claim 8.
